(19) United States
(12) Patent Application Publication
(10) Pub. No.: US 2025/0143991 A1
(43) Pub. Date: May 8, 2025

(54) SULFATE-BASED SURFACTANT FREE ORAL CARE COMPOSITION WITH IMPROVED LOW TEMPERATURE STABILITY

(71) Applicant: Kolmar Korea Co., Ltd., Sejong-si (KR)

(72) Inventors: Hongseok OH, Seoul (KR); Sungmin PARK, Seoul (KR); Hyejin JEON, Seoul (KR)

(73) Assignee: Kolmar Korea Co., Ltd., Sejong-si (KR)

(21) Appl. No.: 18/931,272

(22) Filed: Oct. 30, 2024

(30) Foreign Application Priority Data
Nov. 3, 2023 (KR) 10-2023-0150614

(51) Int. Cl. A61K 8/46; A61K 8/34

(52) U.S. Cl. CPC A61K 8/463; A61K 8/345; A61K 2800/33

(57) ABSTRACT

Disclosed is a sulfate-based surfactant free oral care composition with improved low temperature stability including sodium methyl cocoyl taurate, sorbitol and purified water, and more particularly, an oral care composition with improved formulation stability by adjusting a weight mixing ratio of sorbitol and purified water as a solvent to 1:0.9 to 1:1.72. According to the present invention, it is possible to provide a sulfate based surfactant-free oral care composition including a mild cleansing ingredient having a sulfate-free formulation by using sodium methyl cocoyl taurate as a surfactant. Further, it is possible to improve low temperature stability below approximately 5° C. by adjusting a weight mixing ratio of sorbitol and purified water as a solvent.

### CROSS-REFERENCE TO RELATED APPLICATIONS

This application claims priority to and the benefit of Korean Patent Application No. 10-2023-0150614 filed in the Korean Intellectual Property Office on Nov. 3, 2023, the entire contents of which are incorporated herein by reference.

### TECHNICAL FIELD

The present invention relates to a sulfate-based surfactant free oral care composition including sodium methyl cocoyl taurate, sorbitol and purified water, and more particularly, to an oral care composition with improved formulation stability by adjusting a weight mixing ratio of sorbitol and purified water as a solvent to 1:0.9 to 1:1.72.

### BACKGROUND ART

Conventional oral care compositions have mainly used sulfate-based surfactants such as sodium lauryl sulfate (SLS). However, sulfate-based surfactants, including sodium lauryl sulfate, generally have a problem of being highly irritating. Accordingly, there is a growing demand of consumers for sulfate-free formulations having cleansing and foaming properties while mitigating strong irritation.

Accordingly, there are attempts to utilize sodium methyl cocoyl taurate (SMTC), a mild cleansing ingredient, as a surfactant. However, when sodium methyl cocoyl taurate is applied to existing toothpaste formulations, a stability problem occurs at low temperatures, particularly below 5° C. For example, there are problems in phases of the composition, such as the observation of haze, precipitation, or the like.

Accordingly, the present inventors completed the present invention while conducting research to improve a stability problem at a low temperature while including sodium methyl cocoyl taurate as a surfactant.

Currently, Korean Patent Registration No. 10-2337307 discloses a low irritating oral composition containing a glutamate-based surfactant and an amidopropyl betaine-based surfactant, without a sulfate-based surfactant. Korean Patent Publication No. 10-2011-0026697 discloses a low irritating oral composition including an amide-based nonionic surfactant, sodium lauryl sulfate, a taurate-based anionic surfactant, and a betaine-based amphoteric surfactant. However, there is not disclosed a sulfate-based surfactant free oral care composition including sodium methyl cocoyl taurate, sorbitol and purified water of the present invention.

### PRIOR ARTS

### Patent Documents

(Patent Document 0001) Korean Patent Registration No. 10-2337307
(Patent Document 0002) Korean Patent Publication No. 10-2011-0026697

### SUMMARY OF THE INVENTION

The present invention has been made in an effort to provide a sulfate-based surfactant free oral care composition with improved low temperature stability including sodium methyl cocoyl taurate, sorbitol and purified water.

An exemplary embodiment of the present invention provides a sulfate-based surfactant free oral care composition with improved low temperature stability including sodium methyl cocoyl taurate, sorbitol and purified water.

According to exemplary embodiments of the present invention, it is possible to provide a sulfate-based surfactant free oral care composition with improved low temperature stability including a mild cleansing ingredient having a sulfate-free formulation by using sodium methyl cocoyl taurate as a surfactant.

Further, it is possible to improve low temperature stability below approximately 5° C. by adjusting a weight mixing ratio of sorbitol and purified water as a solvent.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a photograph observing a change in phase of a sulfate-based surfactant free oral care composition with improved low temperature stability prepared according to an example of the present invention.

FIGS. 2 to 5A and 5B are photographs showing changes in phase at a low temperature (5° C.) of a sulfate-based surfactant free oral care composition with improved low temperature stability prepared according to Experimental Example of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, the present invention will be described in more detail through exemplary examples. These examples are to explain the present disclosure in more detail, and it will be apparent to those skilled in the art that the scope of the present disclosure is not limited by these examples in accordance with the gist of the present disclosure.

The present invention provides a sulfate-based surfactant free oral care composition with improved low temperature stability including sodium methyl cocoyl taurate, sorbitol and purified water.

In the present invention, the sodium methyl cocoyl taurate (SMCT) is a type of coconut-derived anionic surfactant, which refers to a sodium salt component of coconut fatty acid amide of N-methyl taurine, and may have excellent foaming property, wetting property, emulsifying property, and softening property in a wide pH range, but is not limited thereto.

In the present invention, the sodium methyl cocoyl taurate may be included in an amount of 1.0 to 1.5 wt % based on the total weight of the composition, but is not limited thereto. At this time, if the sodium methyl cocoyl taurate is less than 1.0 wt %, the surfactant content may be too small, which does not sufficiently perform a cleansing function as the surfactant, and if the sodium methyl cocoyl taurate is more than 1.5 wt %, there may be problems in incompatibility with other ingredients and stability.

In the present invention, the sorbitol is a type of hexahydric alcohol obtained by reducing hexoses such as glucose and has a sweet taste similar to sugar. The sorbitol is white crystal that is flammable and hygroscopic, and is sweet, and easily soluble in water or alcohol, and is used in cosmetic lotions, creams, toothpaste, food additives, synthetic raw materials for vitamin C, sweeteners for diabetic patients, diuretics, etc. The sorbitol has melting points of 93 to 97.5° C., and may have Chemical Formula of CH₂OH·(CHOH)₄CH₂OH.

In the present invention, the sorbitol may function as a solvent, a moisturizer, and a sweetener in the oral care composition, but is not limited thereto.

In the present invention, a weight mixing ratio of the sorbitol and purified water may be 1:0.9 to 1:1.72, but is not limited thereto. At this time, when the weight mixing ratio of the sorbitol and purified water is less than 0.9, there may be a problem in which the stability of a low temperature phase of the oral care composition is reduced, and when the weight mixing ratio of the sorbitol and purified water is more than 1.72, a problem of decreasing the viscosity of the oral care composition may occur.

In the present invention, the oral care composition may further include polyethylene glycol (PEG) having a molecular weight of 500 to 5,000 as a dispersion stabilizer, preferably polyethylene glycol having a molecular weight of 1,500 (PEG 1500), but is not limited thereto. At this time, if the molecular weight of the polyethylene glycol is less than 500, its effectiveness as a dispersion stabilizer is minimal, and if the molecular weight of the polyethylene glycol is more than 5,000, there may be a problem that it is difficult to be evenly dissolved when preparing toothpaste and oral care compositions may occur.

In the present invention, the polyethylene glycol may be included in an amount of 0.01 to 10 wt %, preferably 0.1 to 5 wt %, and more preferably 1.0 to 3.0 wt % based on the total weight of the oral care composition, but is not limited thereto.

In the present invention, the oral care composition may have a viscosity of 800,000 to 1,800,000 cPs under a condition of 25° C., preferably 830,000 to 1,750,000 cPs, and more preferably 850,000 to 1,700,000 cPs, but is not limited thereto.

In the present invention, the oral care composition may have a viscosity of 800,000 to 1,800,000 cPs under a condition of 5° C., preferably 850,000 to 1,750,000 cPs, and more preferably 900,000 to 1,700,000 cPs, but is not limited thereto.

Hereinafter, the present disclosure will be described in detail by Examples. However, the following Examples are just illustrative of the present disclosure, and the contents of the present disclosure are not limited to the following Examples.

<Preparation of Sulfate-Based Surfactant Free Oral Care Composition with Improved Low Temperature Stability>

### EXAMPLE

A sulfate-based surfactant free oral care composition with improved low temperature stability was prepared according to ingredients and contents described in Table 1 below, and a weight mixing ratio of sorbitol and purified water was shown in Table 2 below.

TABLE 1

| Classification | Ex. 1 | Ex. 2 | Ex. 3 | Ex. 4 | Ex. 5 | Ex. 6 | Ex. 7 |
|---|---|---|---|---|---|---|---|
| Purified water | 43.05 | 39.55 | 39.7 | 39.4 | 37.05 | 46.55 | 49.35 |
| Sorbitol | 35 | 38.5 | 38.5 | 38.5 | 41 | 31.5 | 28.7 |
| Sodium methyl cocoyl | 1.2 | 1.2 | 1.05 | 1.35 | 1.2 | 1.2 | 1.2 |
| taurate | | | | | | | |
| PEG 1500 | 1.5 | 1.5 | 1.5 | 1.5 | 1.5 | 1.5 | 1.5 |
| Sodium carboxymethyl | 1.2 | 1.2 | 1.2 | 1.2 | 1.2 | 1.2 | 1.2 |
| cellulose | | | | | | | |
| Silica | 14 | 14 | 14 | 14 | 14 | 14 | 14 |
| Sodium pyrophosphate | 1.5 | 1.5 | 1.5 | 1.5 | 1.5 | 1.5 | 1.5 |
| Sodium | 0.25 | 0.25 | 0.25 | 0.25 | 0.25 | 0.25 | 0.25 |
| monofluorophosphate | | | | | | | |
| Sodium fluoride | 0.1 | 0.1 | 0.1 | 0.1 | 0.1 | 0.1 | 0.1 |
| Flavor | 2 | 2 | 2 | 2 | 2 | 2 | 2 |
| Sodium saccharin | 0.2 | 0.2 | 0.2 | 0.2 | 0.2 | 0.2 | 0.2 |
| hydrate | | | | | | | |

(Unit: wt %)

TABLE 2

| Classification | Ex. 1 | Ex. 2 | Ex. 3 | Ex. 4 | Ex. 5 | Ex. 6 | Ex. 7 |
|---|---|---|---|---|---|---|---|
| Purified water/ | 1.23 | 1.03 | 1.03 | 1.02 | 0.90 | 1.48 | 1.72 |
| sorbitol ratio | | | | | | | |

Oral care compositions prepared according to ingredient contents of Examples 1 to 7 above were evaluated for appearance, viscosity at 5° C., viscosity at 25° C., foaming ability, and taste preference, and the results were confirmed as shown in Table 3 below.

Meanwhile, the viscosity was measured under conditions of RV 7 pin and 1 RPM, and the appearance was evaluated by checking whether there was any abnormality in a phase after storing the composition at 5° C. for one week. If a change in the phase was observed, it was marked as X, and if no change in the phase was observed, it was marked as O. In addition, the foaming ability and taste preference were evaluated based on 5.0-point evaluation, which meant that 1 point was very bad and 5 point was very good.

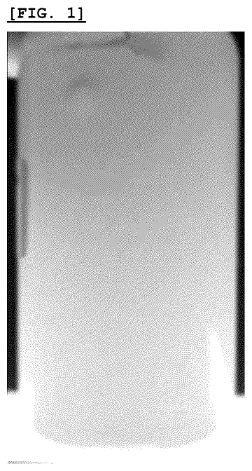

As a result of the evaluation, it was confirmed that all of the oral care compositions according to Examples 1 to 7 had stable phase and viscosity at low temperatures, and showed excellent foaming ability and taste preference evaluations. In addition, as shown in FIG. 1, it was confirmed that the composition of Example 1 was uniformly mixed without any change in phase even at a low temperature.

TABLE 3

| Classification | Ex. 1 | Ex. 2 | Ex. 3 | Ex. 4 | Ex. 5 | Ex. 6 | Ex. 7 |
|---|---|---|---|---|---|---|---|
| Appearance | ◯ | ◯ | ◯ | ◯ | ◯ | ◯ | ◯ |
| Viscosity at | 1,160,000 | 1,180,000 | 1,150,000 | 1,120,000 | 1,440,000 | 1,050000 | 988,000 |
| 5° C. (cPs) | | | | | | | |
| Viscosity at | 1,400,000 | 1,200,000 | 1,320,000 | 1,210,000 | 1,660,000 | 894,000 | 873,000 |
| 25° C. (cPs) | | | | | | | |
| Foaming ability | 4 | 4 | 3 | 5 | 4 | 3 | 3 |
| Taste Preference | 4 | 4 | 4 | 4 | 4 | 3 | 2 |

### Experimental Example 1. Evaluation of Low Temperature Stability, Foaming Ability, Etc. According to Content of Sodium Methyl Cocoyl Taurate

For this experiment, compositions of Comparative Examples 1 and 2 were prepared. The compositions of Comparative Examples 1 and 2 had only differences in the sodium methyl cocoyl taurate content and the ratio of purified water and sorbitol, as shown in Tables 4 and 5 below, but were prepared with the same contents of other ingredients as in Example 1.

TABLE 4

| | Comparative | Comparative |
|---|---|---|
| Classification | Example 1 | Example 2 |
| Purified water | 39.85 | 39.15 |
| Sorbitol | 38.5 | 38.5 |
| Sodium methyl cocoyl taurate | 0.9 | 1.6 |
| PEG 1500 | 1.5 | 1.5 |
| Sodium carboxymethyl cellulose | 1.2 | 1.2 |
| Silica | 14 | 14 |
| Sodium pyrophosphate | 1.5 | 1.5 |
| Sodium monofluorophosphate | 0.25 | 0.25 |
| Sodium fluoride | 0.1 | 0.1 |
| Flavor | 2 | 2 |
| Sodium saccharin hydrate | 0.2 | 0.2 |

(Unit: wt %)

TABLE 5

| | Comparative | Comparative |
|---|---|---|
| Classification | Example 1 | Example 2 |
| Purified water/sorbitol ratio | 1.04 | 1.02 |

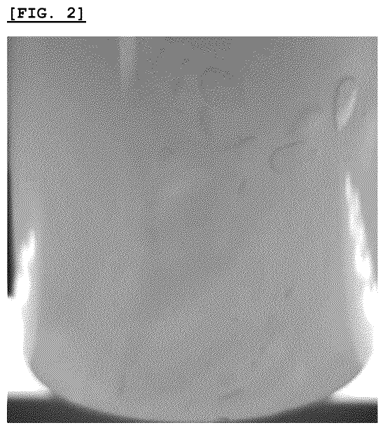

The results of evaluating the appearance after storage at 5° C. for one week, viscosity at 5° C., viscosity at 25° C., foaming ability, and taste preference of the compositions of Comparative Examples 1 and 2 prepared with the contents described in Table 4 above were shown in Table 6 and FIG. 2 below.

TABLE 6

| | Comparative | Comparative |
|---|---|---|
| Classification | Example 1 | Example 2 |
| Appearance | ◯ | X |
| Viscosity at 5° C. (cPs) | 1,020,000 | 1,020,000 |
| Viscosity at 25° C. (cPs) | 1,130,000 | 1,030,000 |
| Foaming ability | 1 | 4 |
| Taste Preference | 2 | 3 |

As shown in Table 6 above, there was no problem when sodium methyl cocoyl taurate was contained in 1.0 to 1.5 wt % based on the weight of the composition, but Comparative Example 1 containing 0.9 wt % of sodium methyl cocoyl taurate was evaluated to have a very low score in foaming ability. That is, in the case of the composition of Comparative Example 1, bubbles were almost not generated, so that the user may not sufficiently feel the sensation of brushing or cleaning. In addition, in the case of Comparative Example 2 containing 1.6 wt % of sodium methyl cocoyl taurate, as shown in FIG. 2, changes in phase were observed in the appearance evaluation after storage for one week at 5° C., as shown in FIG. 2.

### Experimental Example 2. Evaluation of Low Temperature Stability, Foaming Ability, Etc. According to Weight Mixing Ratio of Sorbitol and Purified Water

For this experiment, compositions of Comparative Examples 3 to 5 were prepared. The compositions of Comparative Examples 3 to 5 had only differences in the contents and ratios of sorbitol and purified water, as shown in Tables 7 and 8 below, but were prepared with the same contents of other ingredients as in Example 1.

TABLE 7

| | Com. | Com. | Com. |
|---|---|---|---|
| Classification | Ex. 3 | Ex. 4 | Ex. 5 |
| Purified water | 32.55 | 36.05 | 52.15 |
| Sorbitol | 45.5 | 42 | 25.9 |
| Sodium methyl cocoyl taurate | 1.2 | 1.2 | 1.2 |
| PEG 1500 | 1.5 | 1.5 | 1.5 |
| Sodium carboxymethyl cellulose | 1.2 | 1.2 | 1.2 |
| Silica | 14 | 14 | 14 |
| Sodium pyrophosphate | 1.5 | 1.5 | 1.5 |
| Sodium monofluorophosphate | 0.25 | 0.25 | 0.25 |
| Sodium fluoride | 0.1 | 0.1 | 0.1 |
| Flavor | 2 | 2 | 2 |
| Sodium saccharin hydrate | 0.2 | 0.2 | 0.2 |

(Unit : wt %)

TABLE 8

| Classification | Com. Ex. 3 | Com. Ex. 4 | Com. Ex. 5 |
|---|---|---|---|
| Purified water/sorbitol ratio | 0.72 | 0.86 | 2.01 |

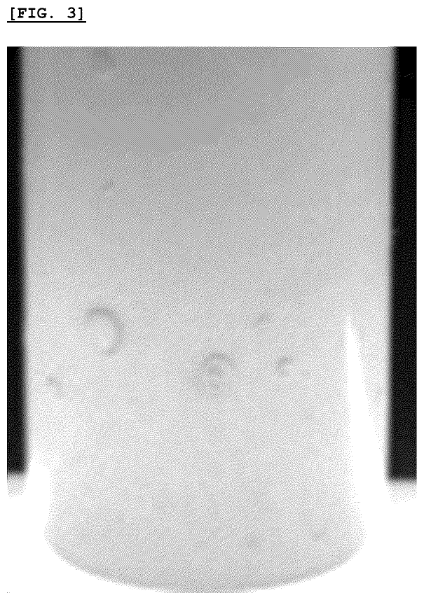

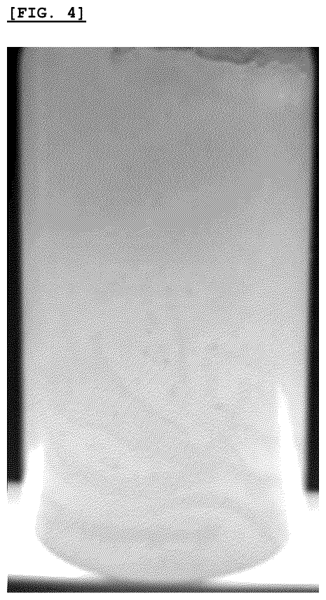

The results of evaluating the appearance after storage at 5° C. for one week, viscosity at 5° C., viscosity at 25° C., foaming ability, and taste preference of the compositions prepared with the contents described in Table 7 above were shown in Table 9 and FIGS. 3 and 4 below.

TABLE 9

| Classification | Com. Ex. 3 | Com. Ex. 4 | Com. Ex. 5 |
|---|---|---|---|
| Appearance | X | X | ◯ |
| Viscosity at 5° C. (cPs) | 1,430,000 | 1,310,000 | 572,000 |
| Viscosity at 25° C. (cPs) | 1,540,000 | 1,480,000 | 468,000 |
| Foaming ability | 4 | 4 | 1 |
| Taste Preference | 4 | 4 | 2 |

When the ratios of purified water/sorbitol according to Examples 1 to 7 above were 0.9 to 1.72, no change in phase was observed even at low temperatures. On the other hand, as shown in FIGS. 3 and 4, in Comparative Examples 3 and 4, where the ratios of purified water/sorbitol were 0.72 and 0.86, as a result of evaluating the appearance after storage at 5° C. for one week, changes in phase, such as the occurrence of bubbles and haze, were observed. In addition, it was confirmed that the composition of Comparative Example 5, in which the ratio of purified water/sorbitol was 2.01, had very low viscosity and received low scores even in evaluation items of foaming ability and taste preference.

### Experimental Example 3. Evaluation of Low Temperature Stability and Foaming Ability According to Addition of Dispersion Stabilizer PEG1500

For this experiment, a composition of Comparative Example 6 was prepared. As shown in Tables 10 and 11 below, the composition of Comparative Example 6 had only differences in addition of PEG1500 as a dispersion stabilizer and a weight mixing ratio of sorbitol and purified water, but was prepared with the same contents of other ingredients as in Example 4.

TABLE 10

| Classification | Comparative Example 6 |
|---|---|
| Purified water | 40.9 |
| Sorbitol | 38.5 |
| Sodium methyl cocoyl taurate | 1.35 |
| PEG 1500 | 0 |
| Sodium carboxymethyl cellulose | 1.2 |
| Silica | 14 |
| Sodium pyrophosphate | 1.5 |
| Sodium monofluorophosphate | 0.25 |
| Sodium fluoride | 0.1 |
| Flavor | 2 |
| Sodium saccharin hydrate | 0.2 |

(Unit: wt %)

TABLE 11

| Classification | Comparative Example6 |
|---|---|
| Purified water/sorbitol ratio | 1.06 |

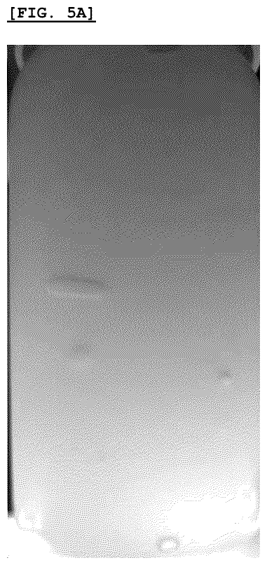

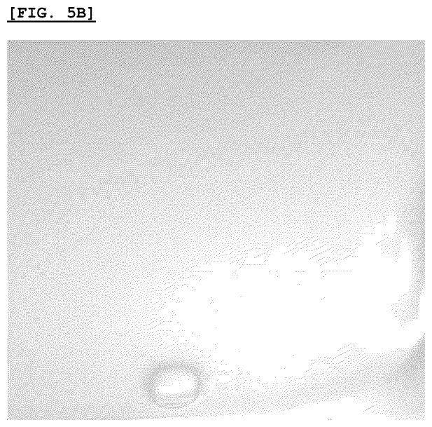

The results of evaluating the appearance, viscosity at 5° C., viscosity at 25° C., foaming ability, and taste preference of the composition prepared with the contents described in Table 10 above were shown in Table 12 and FIGS. 5A and 5B below.

TABLE 12

| Classification | Comparative Example 6 |
|---|---|
| Appearance | X |
| Viscosity at 5° C. (cPs) | 568,000 |
| Viscosity at 25° C. (cPs) | 880,000 |
| Foaming ability | 2 |
| Taste Preference | 4 |

As shown in Table 12 above, there was no problem when PEG1500 was added as a dispersion stabilizer, but in Comparative Example 6 without adding PEG1500 as a dispersion stabilizer, as shown in FIGS. 5A and 5B, changes in phase were observed at a low temperature of 5° C., the viscosity was low, and a low score was confirmed in the foaming ability evaluation. Meanwhile, FIG. 5A is a photograph showing the appearance after storage at 5° C. for one week of the composition of Comparative Example 6, and FIG. 5B is an enlarged photograph of a lower right portion of FIG. 5A.

Hereinafter, exemplary embodiments of the present invention will be described in detail so as to easily implement those skilled in the art. However, the present invention may be implemented in various different forms and is not limited to the exemplary embodiments described herein.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A sulfate-based surfactant free oral care composition with improved low temperature stability comprising sodium methyl cocoyl taurate, sorbitol and purified water.

2. The sulfate-based surfactant free oral care composition with improved low temperature stability of claim 1, wherein the sodium methyl cocoyl taurate is included in an amount of 1.0 to 1.5 wt % based on the total weight of the composition.

3. The sulfate-based surfactant free oral care composition with improved low temperature stability of claim 1, wherein a weight mixing ratio of the sorbitol and purified water is 1:0.9 to 1:1.72.

4. The sulfate-based surfactant free oral care composition with improved low temperature stability of claim 1, further comprising:
polyethylene glycol having a molecular weight of 500 to 2,500.

5. The sulfate-based surfactant free oral care composition with improved low temperature stability of claim 4, wherein the polyethylene glycol is included in an amount of 0.01 to 10 wt % based on the total weight of the composition.

6. The sulfate-based surfactant free oral care composition with improved low temperature stability of claim 1, wherein the oral care composition has a viscosity of 800,000 to 1,800,000 cPs under a condition of 25° C.

7. The sulfate-based surfactant free oral care composition with improved low temperature stability of claim 1, wherein the oral care composition has a viscosity of 800,000 to 1,800,000 cPs under a condition of 5° C.
